(19) United States
(12) Patent Application Publication
(10) Pub. No.: US 2005/0192667 A1
(43) Pub. Date: Sep. 1, 2005

(54) Near vision enhancing intraocular lens

(76) Inventor: Elise N. Schachar, Dallas, TX (US)

Correspondence Address: DOCKET CLERK, P.O. DRAWER 800889, DALLAS, TX, 75380, US

(21) Appl. No.: 11/051,247

(22) Filed: Feb. 4, 2005

Related U.S. Application Data
(60) Provisional application No. 60/541,977, filed on Feb. 5, 2004.

(51) Int. Cl. A61F 2/16

(52) U.S. Cl. 623/6.11

(57) ABSTRACT

An intraocular lens assembly is disclosed that increases the positive spherical aberration of an eye in order to enhance both near vision and distance vision in the eye. The intraocular lens assembly comprises an intraocular lens that has a convex anterior surface and a concave posterior surface that increase the spherical aberration of the eye. The intraocular lens is constructed of a biologically compatible material. The intraocular lens may be used as a replacement for the crystalline lens of the eye. The intraocular lens may be placed within an anterior chamber or a posterior chamber of an a phakic eye or a phakic eye.

### CROSS-REFERENCE TO RELATED APPLICATION

This application claims benefit and priority (under 35 U.S.C. §119(e)) to prior U.S. provisional application Ser. No. 60/541,977 filed on Feb. 5, 2004, and which is incorporated herein by reference.

### TECHNICAL FIELD OF THE INVENTION

The present invention generally relates to intraocular lenses and, more specifically, to intraocular lenses that correct both distance vision and near vision by increasing the positive spherical aberration of the eye.

### BACKGROUND OF THE INVENTION

An intraocular lens is surgically implanted within an eye to replace the optical power that is lost when the crystalline lens of an eye is removed. It may be necessary to remove the crystalline lens of an eye because the crystalline lens has become damaged or diseased. After the crystalline lens has been surgically removed from the eye, an intraocular lens is surgically implanted in place of the crystalline lens.

Prior art intraocular lenses typically correct for distance vision but do not correct for near vision. Multiple intraocular lenses have been designed in an attempt to match the accommodative amplitude of a young phakic eye. These attempts have not been successful. Generally, the prior art accommodating intraocular lenses preclude the pseudophake from reading fine print because the prior art accommodating intraocular lenses are capable of providing only approximately one diopter (1.0 D) of accommodation.

Prior art multifocal intraocular lenses operate using multiple diffractive rings or diffractive annular zones. However, the multiple diffractive rings or diffractive annular zones diffract light. The diffraction creates problems with night vision. The diffraction also creates problems with contrast sensitivity.

Therefore, there is a need in the art for an intraocular lens that is capable of enhancing near vision without using multiple diffractive rings or diffractive annular zones. There is a need in the art for an intraocular lens that is capable of providing a level of near vision that is greater than that provided by prior art intraocular lenses.

### SUMMARY OF THE INVENTION

To address the above-discussed deficiencies of the prior art, it is a primary object of the present invention to provide an intraocular lens that is capable of enhancing near vision of an eye.

One advantageous embodiment of the invention comprises an intraocular lens assembly that comprises an intraocular lens, a first haptic and a second haptic. The first haptic and the second haptic are attached to the intraocular lens and are used to support the intraocular lens in an eye as replacement for the crystalline lens of the eye.

The intraocular lens is formed with a convex anterior surface and a concave posterior surface. The radius of curvature of the convex anterior surface and the radius of curvature of the concave posterior surface of the intraocular lens are selected so that after the intraocular lens is inserted into an eye the spherical aberration of the eye is increased to enhance near vision.

It is an object of the present invention to provide an intraocular lens that increases positive spherical aberration of an eye.

It is also an object of the present invention to provide an intraocular lens that increases positive spherical aberration of an eye by at least one diopter.

It is yet another object of the present invention to provide an intraocular lens that comprises a convex-concave lens.

It is another of object of the present invention to provide an intraocular lens that enhances both near vision and distance vision of an eye.

The foregoing has outlined rather broadly the features and technical advantages of the present invention so that those skilled in the art may better understand the detailed description of the invention that follows. Additional features and advantages of the invention will be described hereinafter that form the subject of the claims of the invention. Those skilled in the art should appreciate that they may readily use the conception and the specific embodiment disclosed as a basis for modifying or designing other structures for carrying out the same purposes of the present invention. Those skilled in the art should also realize that such equivalent constructions do not depart from the spirit and scope of the invention in its broadest form.

Before undertaking the Detailed Description of the Invention below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like. Definitions for certain words and phrases are provided throughout this patent document, those of ordinary skill in the art should understand that in many, if not most instances, such definitions apply to prior uses, as well as future uses, of such defined words and phrases.

In the description of all of the optical surfaces in the invention the usual standard is used in which: (1) light is assumed to come from the left, and (2) a radius of a convex surface facing left is considered to be positive.

### BRIEF DESCRIPTION OF THE DRAWINGS

For a more complete understanding of the present invention and its advantages, reference is now made to the following description taken in conjunction with the accompanying drawings, in which like reference numerals represent like parts:

FIG. 1 illustrates a plan view of an advantageous embodiment of an intraocular lens assembly of the invention;

FIG. 2 illustrates a side view of the advantageous embodiment of the intraocular lens assembly shown in FIG. 1; and

FIG. 3 illustrates a flow chart showing the steps of an advantageous embodiment of the method of the present invention.

### DETAILED DESCRIPTION OF THE INVENTION

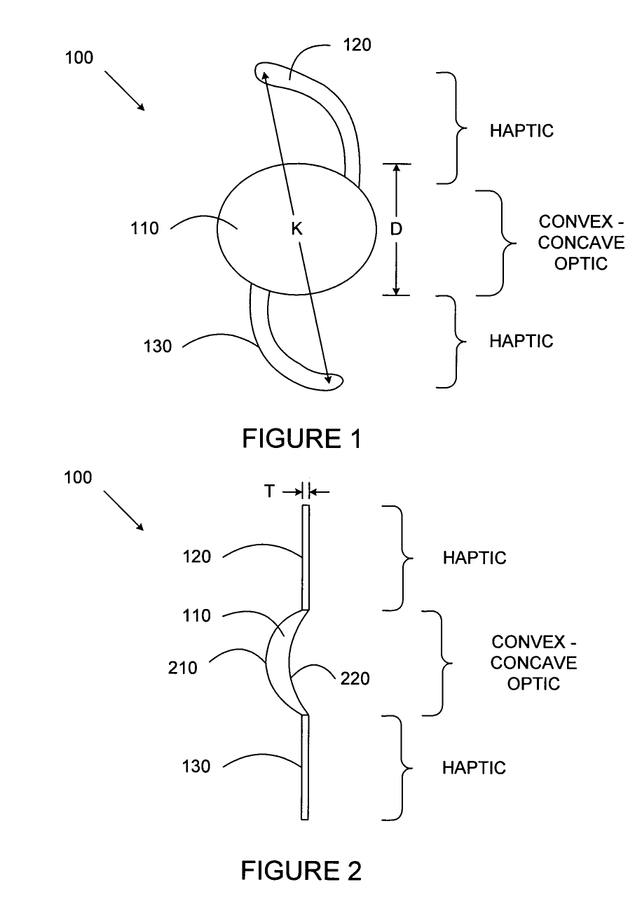

FIGS. 1 through 3 and the various embodiments used to describe the principles of the present invention in this patent document are by way of illustration only and should not be construed in any way to limit the scope of the invention. Those skilled in the art will understand that the principles of the present invention may be implemented in any type of suitably arranged intraocular lens assembly.

FIG. 1 illustrates a plan view of an advantageous embodiment of an intraocular lens assembly 100 of the invention. FIG. 2 illustrates a side view of an advantageous embodiment of an intraocular lens assembly 100 of the invention. The intraocular lens assembly 100 comprises an intraocular lens 110 (also referred to as an optic 110), a first haptic 120 and a second haptic 130.

The intraocular lens 110 is constructed of a biologically compatible material. In one advantageous embodiment the intraocular lens 110 is constructed of polymethylmethacrylate (PMMA) having a positive optical power. In another advantageous embodiment the intraocular lens 110 is constructed of a biologically inert copolymer of acrylate and methylacrylate. The intraocular lens 110 may also be constructed of silicone, hydrogel, hydrophilic acrylic polymers such as hydroxyethyl methacrylate, polysiloxane, and other similar types of biologically inert materials. The intraocular lens 110 may be fashioned using injection molding or lathe cutting techniques.

In one advantageous embodiment the intraocular lens 110 is constructed of a material that is capable of being folded. In another advantageous embodiment the intraocular lens 110 is constructed of a material that is capable of being injected into an eye.

The first haptic 120 and the second haptic 130 are attached to the intraocular lens 110. The first haptic 120 and the second haptic 130 are used to support the intraocular lens 110 in the crystalline lens capsule of the eye or ciliary sulcus. The first haptic 120 and the second haptic 130 may be constructed of polypropylene or polymethylmethacrylate (PMMA). The first haptic 120 and the second haptic 130 shown in FIG. 1 and in FIG. 2 are exemplary structures for securing the intraocular lens 110 within an eye. The intraocular lens 110 may be secured within the crystalline lens capsule of an eye by any suitable type of structure.

In an alternate embodiment of the invention, the first haptic 120 and the second haptic 130 and the intraocular lens 110 are formed as a unitary body. In the alternate embodiment the first haptic 120 and the second haptic 130 are not separate elements that are connected to the intraocular lens 110. Instead, the first haptic 120 and the second haptic 130 are formed from the same piece of lens material as the intraocular lens 110.

The diameter D for the intraocular lens 110 is shown in FIG. 1. A typical value for the diameter D is approximately five millimeters (5.0 mm). The thickness T of the first haptic 120 is shown in FIG. 2. A typical value for the thickness T is approximately one half of a millimeter (0.5 mm). The second haptic 130 has the same thickness T as the first haptic 120. The distance K between the end of the first haptic 120 and the end of the second haptic 130 is shown in FIG. 1. A typical value for the distance K is approximately twelve millimeters (12.0 mm).

As shown in FIG. 2, the anterior surface 210 of intraocular lens 110 has a convex surface. The radius of curvature of the convex anterior surface 210 is designated as “rₐ”. As also shown in FIG. 2, the posterior surface 220 of intraocular lens 110 has a concave surface. The radius of curvature of the concave posterior surface 220 is designated as “rₚ”. Because the intraocular lens 110 has one convex surface 210 and one concave surface 220, the intraocular lens 110 may also be referred to as a “convex-concave optic 110.”

The convex anterior surface 210 and the concave posterior surface 220 of the intraocular lens 110 are selected so that after the intraocular lens 110 is inserted into an eye the spherical aberration of the eye is increased to enhance near vision. The radius of curvature rₐ of the convex anterior surface 210 and the radius of curvature rₚ of the concave posterior surface 220 are selected so that the absolute value of the shape factor (designated “q”) of the intraocular lens 110 has a large value. The value of the shape factor “q” is given by the equation:
q=(rₚ+rₐ)/(rₚ−rₐ) (1)

The radius of curvature rₐ of the convex anterior surface 210 and the radius of curvature rₚ of the concave posterior surface 220 are also selected to minimize the thickness of the intraocular lens 110 and still obtain a desired level of positive spherical aberration.

The values of the radii of curvature rₐ and rₚ are selected so that the phakic eye or pseudophakic eye will have a longitudinal spherical aberration postoperatively that preferably is greater than one diopter (1.0 D) with an approximate pupil diameter of four millimeters (4.0 mm). The value of spherical aberration of the intraocular lens 110 is determined from standard optical equations that relate the shape factor (“q”), the index of refraction (“n”) of the lens material, the thickness of the intraocular lens 110, position length, paraxial focal length, and radius of the pupil. Alternatively, the spherical aberration of the intraocular lens 110 may be determined by optical ray tracing methods, standard computer optical programs, or aberrometer measurements.

An advantageous embodiment of the intraocular lens 110 will now be described. Intraocular lens 110 may have an effective central optical power between ten diopters (10.0 D) and thirty diopters (30.0 D) when it is in the eye and at least one diopter (1.0 D) of positive spherical aberration at two millimeters (2.0 mm) from the optic axis. This may be accomplished by an intraocular lens 110 having a shape factor “q” that is equal to or greater than positive one and one half (q≧+1.5) in order to create the desired amount of spherical aberration.

An intraocular lens 110 with, for example, an index of refraction n=1.49, an anterior radius of curvature rₐ=3.12 millimeters, a posterior radius of curvature rₚ=4.2 millimeters, a central thickness of 0.62 millimeter, and an optical diameter of 5.0 millimeters would increase the longitudinal spherical aberration of the eye more than one diopter (1.0 D) when the pupil is 4.0 millimeters in diameter.

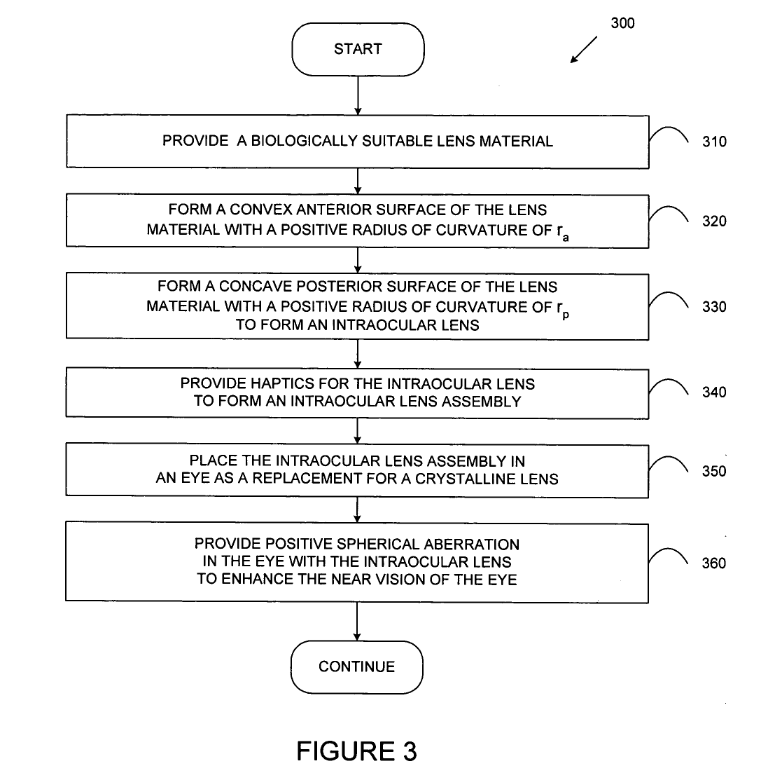

FIG. 3 illustrates a flow chart showing the steps of an advantageous embodiment of the method of the present invention. First provide a biologically suitable lens material of one of the types previously mentioned (step 310). Then form a convex anterior surface of the lens material with a positive radius of curvature rₐ (step 320). Then form a concave posterior surface of the lens material with a positive radius of curvature rₚ to form an intraocular lens (step 330).

Then provide haptics for the intraocular lens to form an intraocular lens assembly (step 340). The haptics may be separate elements that are connected to the intraocular lens or the haptics may be formed together with the intraocular lens as part of unitary body that forms the intraocular lens assembly. Then place the intraocular lens assembly in an eye as a replacement for a crystalline lens (step 350). Then provide positive spherical aberration in the eye with the intraocular lens to enhance the near vision of the eye (step 360).

Although the present invention has been described with an exemplary embodiment, various changes and modifications may be suggested to one skilled in the art. It is intended that the present invention encompass such changes and modifications as fall within the scope of the appended claims.

1. An intraocular lens that increases positive spherical aberration of an eye.

2. The intraocular lens as claimed in claim 1 wherein said intraocular lens increases said positive spherical aberration of said eye by at least one diopter.

3. The intraocular lens as claimed in claim 1 wherein an optical power of said intraocular lens is positive.

4. The intraocular lens as claimed in claim 1 wherein said intraocular lens comprises a convex-concave lens.

5. The intraocular lens as claimed in claim 1 wherein said intraocular lens comprises:
an anterior surface having a positive radius of curvature; and
a posterior surface having a positive radius of curvature.

6. The intraocular lens as claimed in claim 5 wherein said positive radius of curvature of said anterior surface is less than said positive radius of curvature of said posterior surface.

7. The intraocular lens as claimed in claim 5 wherein said intraocular lens has a shape factor “q” that is equal or greater than positive one and one half (+1.5), where said shape factor “q” is given by:
q=(rₚ+rₐ)/(rₚ−rₐ)
where rₐ represents said positive radius of curvature of said anterior surface of said intraocular lens and rₚ represents said positive radius of curvature of said posterior surface of said intraocular lens.

8. The intraocular lens as claimed in claim 5 wherein said positive radius of curvature of said anterior surface is approximately three and twelve hundredths (3.12) millimeters and said positive radius of curvature of said posterior surface is approximately four and two tenths (4.2) millimeters.

9. The intraocular lens as claimed in claim 1 wherein said intraocular lens is made of polymethylmethacrylate (PMMA).

10. The intraocular lens as claimed in claim 1 wherein said intraocular lens is made of one of: silicone and silicone elastomer.

11. The intraocular lens as claimed in claim 1 wherein said intraocular lens is made of one of: biologically inert hydrogel material, biologically inert hydrophilic acrylic polymer, biologically inert polysiloxane, and biologically inert polysulfone.

12. The intraocular lens as claimed in claim 1 wherein said intraocular lens is made of a biologically inert copolymer of acrylate and methacrylate.

13. The intraocular lens as claimed in claim 1 wherein said intraocular lens is made of a material that is capable of being folded.

14. The intraocular lens as claimed in claim 1 wherein said intraocular lens is made of a material that is capable of being injected into an eye.

15. An intraocular lens assembly comprising:
an intraocular lens that increases positive spherical aberration of an eye; and
at least one haptic extending from said intraocular lens for securing said intraocular lens within said eye.

16. The intraocular lens assembly as claimed in claim 15 wherein said intraocular lens and said at least one haptic are made of the same material.

17. The intraocular lens assembly as set forth in claim 15 wherein said intraocular lens comprises a convex-concave lens.

18. The intraocular lens as claimed in claim 15
wherein said intraocular lens comprises an anterior surface having a positive radius of curvature and a posterior surface having a positive radius of curvature; and
wherein said positive radius of curvature of said anterior surface is less than said positive radius of curvature of said posterior surface.

19. A method for increasing positive spherical aberration of an eye, said method comprising the steps of:
providing biologically suitable lens material;
forming a convex anterior surface of said lens material with a positive radius of curvature; and
forming a concave posterior surface of said lens material with a positive radius of curvature to form an intraocular lens.

20. The method as set forth in claim 19 further comprising the steps of:
providing at least one haptic connected to said intraocular lens to form an intraocular lens assembly;
placing said intraocular lens assembly in said eye as a replacement for a crystalline lens; and
providing an increased level of positive spherical aberration of said eye with said intraocular lens.
